(19) United States
(12) Patent Application Publication
(10) Pub. No.: US 2016/0310640 A1
(43) Pub. Date: Oct. 27, 2016

(54) INJECTABLE HIERARCHICAL SCAFFOLDS

(71) Applicant: AARHUS UNIVERSITET, Aarhus C (DK)

(72) Inventors: Jens Vinge Nygaard, Hjortshøj (DK); Christian Perti, Brabrand (DK)

(21) Appl. No.: 15/103,547

(22) PCT Filed: Dec. 10, 2014
(86) PCT No.: PCT/DK2014/050422

§ 371 Date: Jun. 10, 2016

(30) Foreign Application Priority Data
Dec. 11, 2013 (DK) PA 2013 70759

(51) Int. Cl. A61L 27/56; A61L 27/38; A61L 27/18

(52) U.S. Cl. CPC A61L 27/56; A61L 27/18; A61L 27/38

(57) ABSTRACT

The present invention relates to a three-dimensional biocompatible scaffold capable of supporting cell activities, such as growth and differentiation, the scaffold comprising a first biocompatible material and a second biocompatible material, said second material filling a substantial part of the first voids shaped by said first biocompatible material, wherein the scaffold has a diameter of less than 500 μm.

### TECHNICAL FIELD OF THE INVENTION

The present invention relates to three-dimensional injectable scaffolds for tissue engineering and processes of their manufacture and use.

### BACKGROUND OF THE INVENTION

Tissue engineering is a strategy for repairing or regenerating tissue. Cell culture in the context of tissue engineering further requires a three-dimensional scaffold for cell support. A scaffold having a three-dimensional porous structure is a prerequisite in many tissue culture applications, as these cells would otherwise lose their cellular morphology and phenotypic expression in a two-dimensional monolayer cell culture.

An important issue in tissue regeneration and repair is the fabrication of three-dimensional scaffolds in such a manner that they mimic the extracellular matrix and thereby encourage the cells to grow functional tissues and allow the diffusion of nutrients, metabolites and soluble factors.

When regenerating tissue, the properties of the three-dimensional matrix can greatly affect cell adhesion and growth, and determine the quality of the final product. An optimal scaffold material would promote cell adhesion, cell proliferation, expression of cell-specific phenotypes, and the activity of the cells.

Biological materials have been used as scaffolding material in many tissue-engineering applications and within the field of regenerative medicine to control the function and structure of engineered tissue by interacting with transplanted cells. These materials include naturally derived materials such as collagen and alginate. Some biological materials have been proven to support cell ingrowth and regeneration of damaged tissues with no evidence of immunogenic rejection, and encourage the remodelling process by stimulating cells to synthesize and excrete extracellular matrix proteins to aid in the healing process. Extracellular matrix components preserved in these biological materials are also able to influence the phenotypic differentiation of stem cells through specific interactions with e.g. cell surface receptors. However, the usage of such isolated biological materials is limited because of insufficient mechanical properties upon implantation and during perfusion cell seeding.

High porosity of the scaffold is generally recommended to reduce the amount of implanted material and to generate a large surface on to which the cells can adhere. Moreover, interconnectivity, the connection between the pores in the scaffold, is very important since it plays a decisive role in cell mobility within the scaffold and afterwards in the transport (diffusion and convection) of nutrients and cellular waste products.

WO 2010/149176 A1 discloses scaffolds, which can be implanted by open surgery.

A disadvantage of the above described prior art is that it requires surgery to insert such scaffold in the body of a subject, which is often expensive and to great discomfort of the patient especially if the implant is large.

Hence, an improved scaffold would be advantageous, and in particular a more efficient and/or reliable scaffold would be advantageous.

### SUMMARY OF THE INVENTION

Surprisingly the inventors of the present invention have been able to construct small hierarchically constructed scaffolds comprising an internal structure with a controllable stiffness enabling cell differention to desired tissue types, while having an overall stiffness to withstand pressure from surrounding tissue after insertion in the body of a subject.

Though it may at first glance appears as a simple task to produce scaffolds according to the present invention, this is not the case. One challenge during the positioning of the second material within the first material is the solvent used. Since the second material (e.g. PCL) is dissolved in a solvent, said solvent will also dissolve part of the first material (e.g. PCL). This is not a problem for larger scaffolds, where minor degradation of the first material only results in a small percentage of the first material being dissolved, which does not severely compromise the required stiffness of the first material. On the other hand, when the first material is very thin (because of the smaller size), the removal of the same amount of the first material results in a much larger proportion of the first material being dissolved. This, on the other hand may result in a very weak scaffold, which will not have the required strength.

Thus, an object of the present invention relates to providing injectable scaffolds enabling cell differentiation into desired tissue types. In particular, it is an object of the present invention to provide a scaffold that solves the above-mentioned problems of the prior art with avoiding open surgery while maintaining the possibility to support cell differentiation.

It is also an object to provide a scaffold (and a process for producing such scaffolds) which have the stiffness to withstand (and e.g. match) the pressure from the surrounding tissue after injection in the body,

Thus, one aspect of the invention relates to a three-dimensional biocompatible scaffold 1 capable of supporting cell activities, such as growth and differentiation, the scaffold comprising
a) a first biocompatible material 2,
   said first material is shaped as one or more grids forming an open network of first voids, said grid providing protective mechanical support for the second biocompatible material;
b) a second biocompatible material 3,
   said second material being comprised in the first voids shaped by said first biocompatible material 2;
   said second biocompatible material comprising one or more biocompatible polymers;
   said second material being porous and the pores being interconnected;
   said second material having a plurality of open second voids 4a, 4b distributed therein, said open second voids being at least bimodal in size distribution, thereby providing voids which
      allow cells to, optionally infiltrate, grow and differentiate therein, and
      provide a stiffness of the second material different from the stiffness of the first biocompatible material;
         wherein the three-dimensional biocompatible scaffold 1 has a diameter of less than 1500 μm.

Another aspect of the present invention relates to a scaffold according to the present invention for use as a medicament.

Yet an aspect relates to a process for producing an injectable three-dimensional biocompatible scaffold 1 according to the invention, the process comprising:
a) providing a first biocompatible material 2,
   said first material is shaped as one or more grids forming an open network of first voids, said grid providing protective mechanical support for a second biocompatible material 3; wherein the grid has a diameter of less than 1500 μm;
b) preferably, cooling the first biocompatible material to a temperature equal to or below 5° C.;
c) adding a solution comprising one or more biocompatible polymers and two or more solvents to a substantial part of the open network.
d) removing said solvents resulting in a second biocompatible material 3 within the open network,
   said second material being comprised in the first voids shaped by said first biocompatible material;
   said second biocompatible material comprising one or more biocompatible polymers;
   said second material being porous and the pores being interconnected;
   said second material having a plurality of open second voids 4a, 4b distributed therein, said open second voids being at least bimodal in size thereby providing voids which
      allow cells to, optionally infiltrate, grow and differentiate therein, and
      provide a stiffness of the second material different from the stiffness of the first biocompatible material.

In a preferred embodiment, the first biocompatible material is cooled to a temperature equal to or below 5° C.

Yet another aspect of the present invention is to provide a kit comprising
a subset of scaffolds 1 according to the invention; and
means for providing the scaffolds to a subject.

A further aspect relates to the use of scaffolds according to the invention, for in vitro seeding of cells.

### BRIEF DESCRIPTION OF THE FIGURES

FIG. 1 shows a schematic drawing of an injectable scaffold according to one embodiment of the invention. 1: Scaffold; 2: First material; 3: Second material; 4a, 4b: Voids in second material; and 5: Living cells. Thus, it is to be understood that the first material forms the overall structure and protects the scaffold against external forces. The second material provides a stiffness, which allows cells to differentiate into the desired cell type. The larger pores in the second material provide space for living cells, and the smaller pores 4b provides space for diffusion of nutrients and the like.

FIG. 2 shows pictures of FDM produced geometries. A) X-Y direction. B) X-Z direction.

FIG. 3A shows temperatures over time using varying cooling gradients. X axis: Time in 0.1 seconds. Y-axis: Temperature in Kelvin of a mold containing the first material into which the solution of the second material is introduced. FIG. 3B shows specific variations of stiffness prepared in the second biomaterial as a function solvent composition.

FIG. 4 shows two different scaffolds produced by two different cooling gradients.

FIGS. 5-7 show scaffolds according to the invention. The present invention will now be described in more detail in the following.

### DETAILED DESCRIPTION OF THE INVENTION

### Definitions

Prior to discussing the present invention in further details, the following terms and conventions will first be defined:

The pH of blood is usually slightly basic with a value of pH 7.4. This value is referred to as physiological pH in biology and medicine.

Scaffolds

Polymeric scaffolds are intended to function as a temporary extracellular matrix (ECM) until regeneration of bone or other tissues has occurred. Therefore, the more closely it resembles an in vivo microenvironment, the more likely the success of the scaffold.

State-of-the-art polymeric scaffolding has been considered for the formation of 3D implant models that fit into specific defects. This is mainly obtained by fused deposition methods copying a 3D scanning image. Materials used are pure polymers, mixed polymers, or mixture of polymer and e.g. bone-conducting granules. But the precision deposition is restricted by the limited surface area available for cell attachments onto individual fibres.

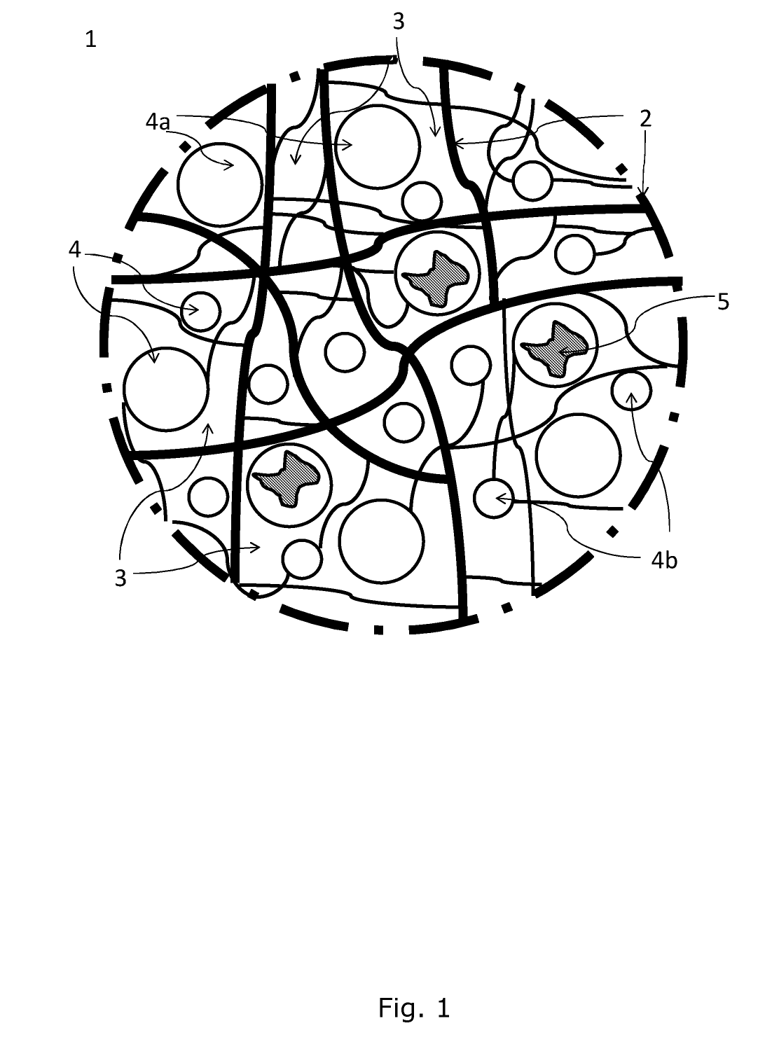

Hence, in the present invention, a combination of the strict 3D structure with an interior providing abundant surface area for cell ingrowth is proposed as a new and reasonable solution. The abundant surface area is obtained by providing a material with a combination of submicrocellular structures and interconnected pores. Submicrocellular structures are defined as having an average cell/pore/void size below about 1 micrometre. In FIG. 1, sub-microcellular voids are illustrated as 4b.

Interconnectivity, the connection between the pores and/or the submicrocellular structures in the scaffold, is very important since it plays a decisive role in the diffusion of cells into the scaffold and the transport of nutrients and cellular waste products. The supermicrocellular structures are too small for the cells to infiltrate, and this allows for a constant and continuous transport of nutrients and cellular waste products within the whole scaffold. Without the supermicrocellular structures, the scaffold could risk clotting with infiltrating cells, resulting in a disruption of transport of nutrients and cellular waste products. This could result in disadvantageous cell death in the central regions of the scaffold. In addition, the internal structures provide an internal stiffness allowing cells to differentiate into desired tissue types based on the stiffness.

The scaffolds according to the present invention are optimized for being injectable. In the present context injectable is to be understood as having a diameter of less than 1500 μm. This corresponds to the diameter of the larger types of syringes, which may be used to inject the scaffolds of the invention into a subject.

Thus, one aspect of the present invention relates to a three-dimensional biocompatible scaffold 1 capable of supporting cell activities, such as growth and differentiation, the scaffold comprising
a) a first biocompatible material 2,
   said first material is shaped as one or more grids forming an open network of first voids, said grid providing protective mechanical support for the second biocompatible material;
b) a second biocompatible material 3,
   said second material being comprised in the first voids shaped by said first biocompatible material 2;
   said second biocompatible material comprising one or more biocompatible polymers;
   said second material being porous and the pores being interconnected;
   said second material having a plurality of open second voids 4 distributed therein, said open second voids being at least bimodal in size distribution 4a, 4b, thereby providing voids which
      allows cells to, optionally infiltrate, grow and differentiate therein, and
      provides a stiffness of the second material different from the stiffness of the first biocompatible material;
         wherein the three-dimensional biocompatible scaffold 1 has a diameter of less than 1500 μm.

Solid freeform fabrication (SFF) is a collection of techniques for manufacturing solid objects by the sequential delivery of energy and/or material to specified points in space to produce a solid. SFF is sometimes referred to as rapid prototyping, rapid manufacturing, layered manufacturing and additive fabrication.

Solvent Casting—Particulate Leaching (SCPL) allows the preparation of porous structures with regular porosity, but with a limited scaffold size. First the polymer is dissolved into a suitable organic solvent (e.g. polylactic acid could be dissolved into dichloromethane), and then the solution is cast into a mould filled with porogen particles. Such porogen can be an inorganic salt like sodium chloride, crystals of saccharose, gelatin spheres or paraffin spheres. The size of the porogen particles will affect the size of the scaffold pores, while the polymer to porogen ratio is directly correlated to the porosity of the final structure. After the polymer solution has been cast the solvent is allowed to fully evaporate, and then the composite structure in the mould is immersed in a bath of a liquid suitable for dissolving the porogen: water in case of sodium chloride, saccharose and gelatin or an aliphatic solvent like hexane for paraffin. Once the porogen has been fully dissolved a porous structure is obtained. Beside the small sized scaffold that can be obtained, another drawback of SCPL is the use of organic solvents which must be fully removed to avoid any possible damage to the cells seeded on the scaffold. Finally, it is the mould that determines the overall scaffold shape in contrary to SFF.

Gas foaming can be used to overcome the necessity of using organic solvents and solid porogens. This is done by using gas as the porogen. First disc shaped structures made of the desired polymer are prepared by means of compression moulding using a heated mould. The discs are then placed in a chamber where they are exposed to high pressure CO₂ for several days. The pressure inside the chamber is gradually restored to atmospheric levels. During this procedure, the pores are formed by the carbon dioxide molecules that abandon the polymer, resulting in a sponge like structure. A problem related to such a technique is caused by the excessive heat used during compression moulding. The excessive heat prohibits the incorporation of any temperature labile material into the polymer matrix. Secondly, large volume changes are accompanied with the pore formation making it difficult to control the final overall scaffold shape without using a mould. Again, introducing a restriction of the scaffold geometry is not present with SFF.

Emulsification combined with lyophilisation/freeze-drying is a technique that does not require the use of a solid porogen like SCPL. First, a synthetic polymer is dissolved into a suitable solvent (e.g. polylactic acid in dichloromethane). Then water is added to the polymeric solution and the two liquids are mixed in order to obtain an emulsion. Before the two phases can separate, the emulsion is cast into a mould and quickly frozen by means of immersion into liquid nitrogen. The frozen emulsion is subsequently freeze-dried to remove the dispersed water and the solvent, thus leaving a solidified, porous polymeric structure. Freeze-drying allows a faster preparation as compared to SCPL, since it does not require a time consuming leaching step.

Thermally Induced Phase Separation (TIPS) is similar to the previous technique; this phase separation procedure requires the use of a solvent that can be solidified by lowering the temperature and which is easy to sublime. Dioxane could for example be used to dissolve polylactic acid. Phase separation is aided e.g. by the addition of a small quantity of water which results in the formation of a polymer-rich and a polymer-poor phase. During phase separation, the mixture is cooled below the solvent melting point and subsequently lyophilized to sublime the solvent, thereby obtaining a porous structure.

The term “biocompatible” is to be understood as, but not limited to, eliciting an acceptable immune response in a given organism. Indeed, since the immune response and repair functions in the body are highly complicated it is not adequate to describe the biocompatibility of a single material in relation to a single cell type or tissue.

The term “compressive stiffness” in relation to the stiffness of the first and the second material according the invention is defined as the rigidity of an object; the extent to which it resists deformation in response to an applied force, for instance a compressive force. In the present context, stiffness is determined by an experimental measurement in which the result is a work curve expressed by the biomaterial as it is subjected to a well-defined force.

In an embodiment, the grid is formed of micron sized strands. The term “micron sized” is to be understood as being in the range 25-350 μm, such as 50-220 μm or such as 75-125 μm.

The term “biodegradable” is to be understood as, but not limited to, the chemical, such as biochemical, breakdown of materials by a physiological environment, such as in a human or animal. Thus, in an embodiment the scaffolds according to the invention are biodegradable.

The term “open cells” is to be understood as, but not limited to, a delimited hollow space wherein the walls or surfaces are broken. It is not to be confused with the basic organizational unit of all living organisms. Hence, “open cells” is not a reference to living matter, e.g. cells isolated from living tissue.

The term “cavity” is to be understood as, but not limited to, both pores, interconnected pores, and “open cells”. Unfortunately, within the engineering literature the words cavity and pores are referred to as “cells” and is not to be confused with the basic organizational unit of all living organisms.

The scaffold of the present invention has a size which allows injection by e.g. a syringe, while still having a hierarchical structure providing an external stiffness allowing withstanding compressional forces of surrounding tissue, while still allowing controlled differentiation of cells present therein due to the internal stiffness of the scaffold.

Thus, in an embodiment the first material has a value of compression stiffness comparable to the value of compression stiffness of the surrounding tissue after insertion in a body. In another embodiment, the first material has a compression stiffness able to withstand the compressive forces from the surrounding targeted tissue after insertion in a body.

Charge of Polymers

The charge of the biocompatible polymers may be able to assist in cell attachment. Thus, in yet an embodiment the one or more biocompatible polymers are negatively charged at physiological pH. In a further embodiment, the one or more biocompatible polymers are positively charged at physiological pH.

Cells in Scaffold

The scaffold according to the present invention may be infiltrated by cells after insertion, however in some situations it may be advantageous if the scaffolds a seeded with living cells 5. Thus, in an embodiment the scaffold further comprises living cells 5. In an embodiment, the cells are stem cells, e.g. obtained from the person in which the scaffolds are to be inserted in. In a further embodiment, the scaffold comprises living cells positioned within the voids 4A of the second biocompatible material 3. In one aspect of the present invention, cells having the capacity to differentiate into lineages of the desired tissue are seeded onto the scaffold. To improve the seeding efficiency, the scaffold may subsequently be coated with biocompatible polymers, thereby encapsulating the cells in the scaffold.

Size of Voids

The size of the voids provided by the first material 2 may vary. Thus, in an embodiment the open network of first voids have a size in the range 25 μm-800 μm, preferably in the range 75 to 500 μm.

Similarly, the size of the second voids provided by the second material may vary. Thus, in an embodiment the open network of (bimodal) second voids comprises average diameters in the range 20-500 μm 4a and sizes in the range 0.1-10 μm 4b. It is of course to be understood that the pores in the second material cannot be larger than the pores in the first material. The porosity also allows for diffusion throughout the scaffold. Diffusion throughout the scaffold is to be understood in the meaning, but not limited to, that substantially the entire scaffold is diffused, such as for example about 99%, such as about 90%, such as about 80%, such as about 70% of the scaffold is diffused. Transport mechanisms (diffusion and convection) can be guided through larger channels with in the scaffold.

As mentioned above, the pores formed by the second material is bimodal in size 4a, 4b. In yet an embodiment the average pore size of the larger pores formed by the second biocompatible material is in the interval of 75 to 800 μm, such as in the range from about 75 to about 500 μm, such as within a range from about 100 to 500 micrometres. In another embodiment the average pore size of the smaller pores 4b formed by the second biocompatible material is in the range 0.01-10 μm, such as in the range 0.1-10 μm, such as within a range 0.1-5 μm such as in the range 1-5 μm. Preferably the average size of the larger pores 4a are in the range 75 to 800 μm and the smaller pores 4b are in the range 0.01-10 μm.

Overall Size of Scaffold

The overall size of the scaffold may vary depending on the tissue in which it is to be inserted and the means for insertion. The size of the injectable scaffold must of course be of a size enabling injection such as by a syringe. However the largest syringes (diameter of around 3 mm) still result in pain to the subject. Thus, it would be advantageous if syringes with a smaller diameter could be used. Thus, in an embodiment the scaffold according to the invention has a maximum diameter in the range 10-1500 μm, such as 100-1000 μm, such as 300-1000 μm, such as 500-1000 μm, such as 100-800 μm, such as 100-500 μm, such as 100-200 μm. An advantage of smaller scaffolds is that they can be inserted by injections. If even smaller scaffolds are produced, they may be inserted by syringes having smaller diameter thereby providing less discomfort to the subject having the scaffolds inserted.

Compressive Stiffness of Selected Tissues

Different tissues have different compressive and tensile stiffness. Table 1 below shows the apparent stiffness of selected tissues, not considering viscoelastic properties.

TABLE 1

| Tissue: | Stiffness | |
|---|---|---|
| Cortical bone (human) | 17 | GPa |
| Trabecular bone (human) | 4 | GPa |
| Tendon | 1.2 | GPa |
| Articular cartilage | 15 | MPa |
| Cornea of the eye | 11 | MPa |
| Muscle | 1 | MPa |
| Arteries | 1 | MPa |
| Liver | 275 | kPa |
| Meniscus | 100 | kPa |
| Adipose (fat) tissue | 50 | kPa |
| Kidney | 45 | kPa |
| Brain | 3 | kPa |
| Lens of the eye | 2.2 | kPa |
| Collagen | 1 | GPa |
| Elastin | 600 | kPa |

Hence, to mimic the mechanical properties of a tissue, it may be contemplated that the scaffold must have at least equal a stiffness as the tissue of interest to avoid strain incompatibility under the local forces provided by the body itself or by external forces. However, the stiffness of the scaffold must at the same time be adequate to direct the lineage of a stem cells to evolve into the correct tissue. It is seen from the work of Engler et al. (Matrix Elasticity Directs Stem Cell Lineage Specification.” Cell 126 (4):677-689, 2006) that the optimal stiffness of a scaffold for generating bone tissue is 34 kPa. However, the compression stiffness of bone lies in the range of 4-17 GPa. Thus, the optimal stiffness of the scaffold may collapse under the local pressure provided by the body itself.

Table 2 below shows the optimal compressive stiffness of the internal structures of the second biocompatible material according to the present invention for differentiating stem cells into the desired tissue type

TABLE 2

| | Optimal compressive | Preferred range of compressive | |
|---|---|---|---|
| | stiffness of second | stiffness of second | |
| Tissue type | biocompatible material | biocompatible material | |
| Bone (human) | 40 kPa | 30-100 | kPa |
| Cartilage | 20 kPa | 15-30 | kPa |
| Muscle | 10 kPa | 5-15 | kPa |
| Brain | 0.36 kPa | 0.2-2 | kPa |

Thus, for optimal scaffolds the following properties of the first and second materials may be required depending on the tissue type (table 3).

TABLE 3

| | | | Preferred range of | |
|---|---|---|---|---|
| | Preferred range of | | compressive stiffness of | |
| Tissue type | stiffness of first material | | second material | |
| Cortical bone | 1-30 | GPa | 30-100 | kPa |
| (human) | | | | |
| Cancellous bone | 2-1000 | MPa | 30-100 | kPa |
| (human) | | | | |
| Cartilage | 1-50 | MPa | 15-30 | kPa |
| Muscle | 1-50 | MPa | 5-15 | kPa |
| Brain | 0.3-100 | kPa | 0.2-2 | kPa |

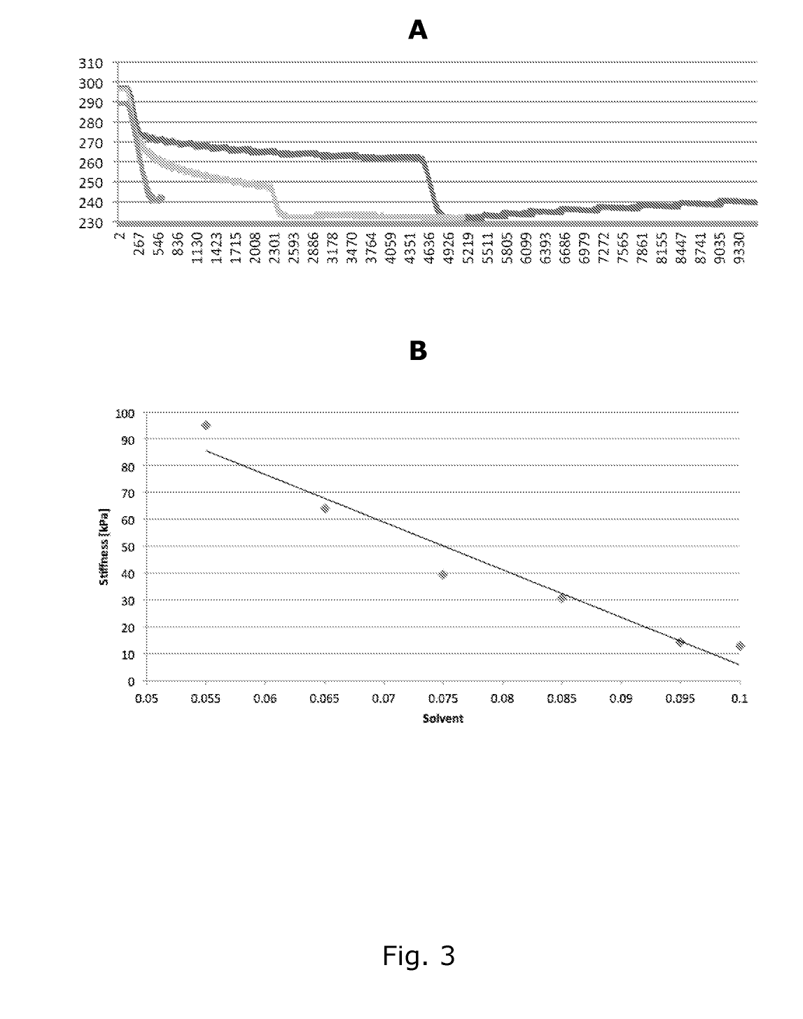

The inventors of the present invention have solved this problem by manufacturing an injectable scaffold comprising a hierarchical structure. FIG. 3B shows that different types of second material 3 with the desired compressive stiffness can be produced using the method of the invention.

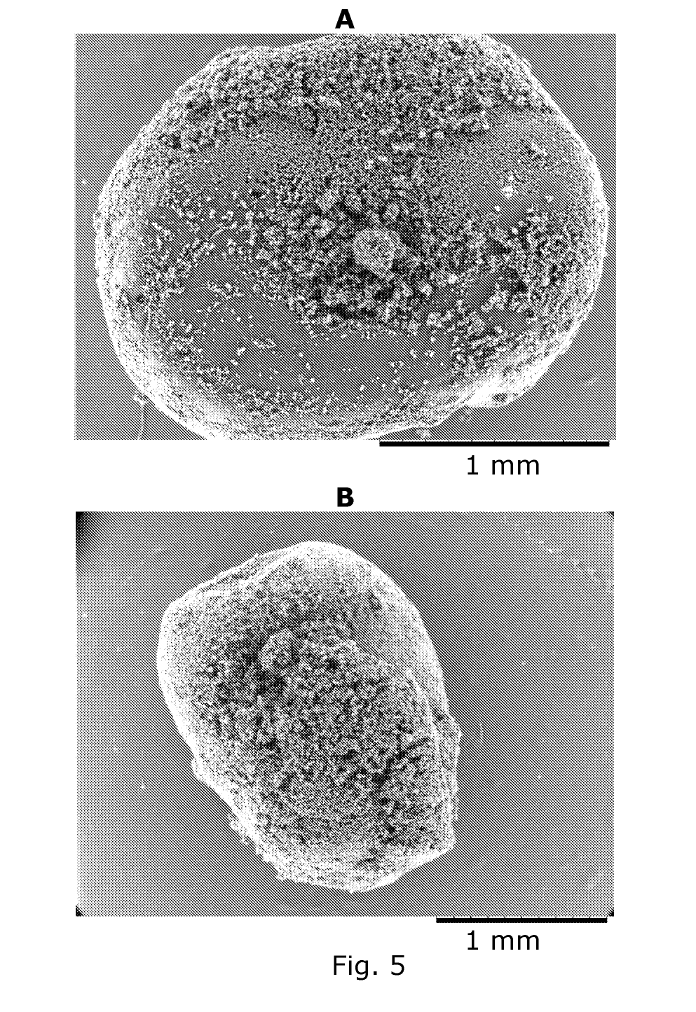

The inventors of the present invention have also made scaffolds wherein the compression stiffness (initial compressive stiffness) of the second biocompatible material 3 of the three-dimensional biocompatible scaffold 1, compression are in the range of 0.31-62 kPa. When combined with the supporting grid comprising a first biocompatible material 2, the compression stiffness was measured in the range of 360-5100 kPa. Mechanical tests on bulk specimens were carried out following ISO 3386-1. FIG. 5, demonstrate by scanning electron microscopy how the second material in embedded into the first. For a specific biocompatible polymer it has been demonstrated how it is possible to control its stiffness through proper selection solvent composition, see example 4 and FIG. 3.

In a preferred embodiment of the present invention, the stiffness of the supporting grid is 5 times higher than the second biocompatible material, such as in the range of 5-100000 times higher, e.g. 10 times higher, such as in the range of 15-90000 times higher, e.g. 50 times higher, such as in the range of 55-90000 times higher, e.g. 100 times higher, such as in the range of 200-50000 times higher, e.g. 500 times higher, such as in the range of 700-30000 times higher, e.g. 900 times higher, such as in the range of 1.500-20.000 times higher than the second biocompatible material.

In one embodiment of the present invention, the scaffold has a comparable value of stiffness to the value of stiffness of the targeted tissue. As an example, the inventors have produced scaffolds with a measured value of compression stiffness of 1 to 72 MPa. Such a scaffold could be useful for reconstruction trabecular bone, cartilage, or muscle tissue, where the target tissue has a comparable value of compression stiffness. A “comparable” value is to be understood as a value not deviating more than 35% from the other value, such as 25%, e.g. 20%, preferably not deviating more than 10%, e.g. 5%.

In another embodiment of the present invention, the scaffold has compatible compression stiffness to withstand the compressive pressure from the surrounding targeted tissue. “Compatible” compression stiffness is to be understood in such a way that the scaffold will remain substantially decompressed when the surrounding targeted tissue presses on the scaffold when injected. The scaffold must not collapse during use. Substantially decompressed is to be understood in such a way that the scaffold volume must not be reduced more than 35% during use, such as 25%, e.g. 20%, preferably not more than 10%, e.g. 5%.

Stiffness of the Second Biocompatible Material

The forces by which a single cell can pull are in the range of nN, and are phenotype specific, (Fu et al. Mechanical Regulation of Cell Function with Geometrically Modulated Elastomeric Substrates.” Nature Publishing Group 7 (9) (August 1): 733-736. 2010). Engler et al. (Matrix Elasticity Directs Stem Cell Lineage Specification.” Cell 126 (4) (August): 677-689. 2006) studied the various cell reactions correlated to changes in the stiffness of a flat substrate and thereby demonstrating cell dependence on mechanical interaction. Specifically, he showed by microarray profiling, that substrate stiffness close to 1, 11, and 34 kPa can specify the lineage of a mesynchemal stem cell and commit it to respectively the phenotypes of neurons, muscle, or bone. Hence, it is possible to adjust the stiffness of the second biocompatible material of the present invention to the tissue type it is desired to obtain.

As mentioned above the second material 3 provides both the stiffness which allows cells to differentiate into a desired tissue type, while also allowing enough space for the cells to infiltrate grow and differentiate. In an embodiment the second biocompatible material has a compression stiffness in the range of 0.3-100 kPa. As can be seen from table 2, stiffness's in these ranges allow differentiation into different tissue types.

In the present case “compressive stiffness” is to be understood as a measure of the resistance offered by an elastic body to deform, and is also known as the modulus of elasticity. Stiffness may be determined by tensile or compressive tests conducted on the biomaterial or tissue of interest.

Stiffness of the First Biocompatible Material

As mentioned above the first material provides an overall stiffness of the scaffolds to withstand the forces of the surrounding tissue after insertion. Thus, in an embodiment the first biocompatible material 2 has a compressive stiffness in the range of 1 kPa-20 GPa. As can be seen from table 1, stiffness's in this range allows the scaffold to withstand the forces from the surrounding tissues. An advantage of not just having a very high stiffness of the first material is that by adapting the stiffness to the surrounding tissue, “thinner” and/or lighterfirst material 2 may be used for certain tissues allowing more space for the second material. However, in principle a very stiff first material 2 may be used for all tissue types and thus only varying the compressive stiffness of the second material 3 according to the desired tissue type the cells are to differentiate into.

It is to be understood that pores/voids in the second material may be uniformly distributed in said second material or substantially uniformly distributed in said second material.

In the following list of embodiment's different preferred combinations of stiffness's are provided.

In an embodiment the first biocompatible material 2 has a compression stiffness in the range of 0.3-100 kPa and the second biocompatible material 3 has a compression stiffness in the range of 0.3-100 kPa. Such combination may be desirable for soft tissue types such as kidney, brain, eye lens, and/or adipose.

In another embodiment the first biocompatible material 2 of the three-dimensional biocompatible scaffold 1 has a compression stiffness in the range of 60 kPa-2 MPa and the second biocompatible material 3 has a compression stiffness in the range of 0.3-100 kPa. Such combination may be desirable for tissue types such as meniscus, liver, veins, arteries, and/or muscle.

In a further embodiment the first biocompatible material 2 of the three-dimensional biocompatible scaffold 1 has a compression stiffness in the range of 2-1000 MPa and the second biocompatible material 3 has a compression stiffness in the range of 0.3-100 kPa. Such combination may be desirable for the following tissue types: cornea and cartilage.

In yet a further embodiment the first biocompatible material 2 of the three-dimensional biocompatible scaffold 1 has a compression stiffness in the range of 1-25 GPa and the second biocompatible material 3 has a compression stiffness in the range of 0.3-100 kPa. Such combination may be desirable for the following tissue types: bone, cartilage, or tendon.

The compression stiffness may be also be expressed relative to each other. Thus, in an embodiment the compression stiffness of the supporting grid (first material, 2) is at least 5 times higher than the second biocompatible material, such as in the range of 5-300000 times higher, such as in the range of 15-90000, such as in the range of 55-90000, such as in the range of 200-50000, such as in the range of 700-30000, such as in the range of 1500-20000, such as in the range of 5-100 times higher than the second biocompatible material.

Materials for First Biocompatible Material

The material of the first biocompatible material may vary. In an embodiment the first biocompatible material comprises material selected from the group consisting of PCL, PET, PC, PEEK, PP, PE, and their derivatives.

Materials for Second Biocompatible Material

The material of the second biocompatible material may vary. Thus, in yet an embodiment the second biocompatible material comprises material selected from the group consisting of Polyethyleglycol (PEG), Polylacticglycolacid (PLGA), Poly Lactic Acid (PLA), Poly Lactic Lactic Acid (PLLA), Polycaprolactone (PCL), Polyethyleterapthalate (PET), Polycarbonate (PC), Polyetheretherketone (PEEK), Polypropylene (PP), Polyethylene (PE), and their derivatives.

In a preferred embodiment both the first and the second material is PCL.

Coatings

If cells are positioned in the scaffold before insertion, the scaffold may be improved by different coating layers. Thus, in yet an embodiment the scaffold further comprises a first biocompatible polymer coating, said first biocompatible polymer coating comprising one or more biocompatible polymers being positively charged at physiological pH, wherein living cells are positioned in-between the second biocompatible material and the first biocompatible polymer coating. In yet a further embodiment the scaffold comprises a second biocompatible polymer coating, said second biocompatible polymer coating comprising one or more biocompatible polymers being negatively charged at physiological pH, said second biocompatible polymer coating positioned on top of the first biocompatible polymer coating.

In another embodiment the scaffold comprises a first biocompatible polymer coating, said first biocompatible polymer coating comprising one or more biocompatible polymers being negatively charged at physiological pH, said living cells being positioned in-between the second biocompatible material and the first biocompatible polymer coating. In another embodiment the scaffold comprises a second biocompatible polymer coating, said second biocompatible polymer coating comprising one or more biocompatible polymers being positively charged at physiological pH, said second biocompatible polymer coating being positioned on top of the first biocompatible polymer coating.

In an additional embodiment the second biocompatible material is coated with a first biocompatible polymer coating comprising one or more biocompatible polymers being positively charged at physiological pH, wherein a second biocompatible polymer coating comprising one or more biocompatible polymers being negatively charged at physiological pH and being positioned on top of the first biocompatible polymer coating, wherein cells being positioned in-between the first and second biocompatible polymer coatings.

In yet an additional embodiment the second biocompatible material is coated with a first biocompatible polymer coating comprising one or more biocompatible polymers being negatively charged at physiological pH, wherein a second biocompatible polymer coating comprising one or more biocompatible polymers being positively charged at physiological pH and being positioned on top of the first biocompatible polymer coating, wherein cells being positioned in-between the first and second biocompatible polymer coatings.

In an embodiment, the scaffold surface is coated with a natural or synthetic coating material such as protein, peptides, nucleotides, and/or small interfering RNAs.

Cell Density and Volume of Said Second Open Cells/Voids

The number of open voids in the scaffold may vary. Thus, in an embodiment the cell density of said second open cells in said second material is lying in a range from about 10⁹ to about 10¹⁵ open cells per cubic centimetre of said second material. To guarantee good interconnectivity, the cell density of said cells/voids in said second material in a preferred embodiment of the present invention is lying in a range from about 10⁹ (ten to the ninth power) to about 10²⁰ cells per cubic centimetre of said second material, such as from about 11¹⁰ to about 10¹⁸, more preferably such as from about 12¹⁰ to about 10¹⁵ cells per cubic centimetre of said second material.

In one embodiment of the present invention, the average size of said cells/voids being from about 1 nanometre to about 6 micrometres, such as from about 1 nanometre to about 5 micrometres, such as from about 1 nanometre to about 4 micrometres, such as from about 1 nanometre to about 2 micrometres, such as from about 1 nanometre to about 1 micrometre, such as from about 1 nanometre to about 900 nanometres, such as from about 5 nanometres to about 800 nanometres, such as from about 10 nanometres to about 700 nanometres, such as from about 15 nanometres to about 600 nanometres, such as from about 15 nanometres to about 500 nanometres, such as from about 15 nanometres to about 300 nanometres, such as from about 20 nanometres to about 100 nanometres, preferably such as from about 25 nanometres to about 50 nanometres.

In yet an embodiment the total volume of the open cells 4a, 4b formed in said second material comprises a fractional percentage of the total volume of said second material 3, which lies within a range from about 20 to about 90 fraction percentage. In another embodiment, the total volume of the cells/voids in said second material comprise a fractional percentage of the total volume of said second material which lies within a range from about 10 to about 99 fraction percent, such as from about 15 to about 98, such as from about 20 to about 95, more preferably such as from about 50 to about 90 fraction percent.

Medical Use

The scaffolds according to the present invention may be degraded during use due to biodegradability. Thus, an aspect the present invention relates to the scaffold according to the invention for use as a medicament.

In yet an aspect the present invention relates to the scaffold according to the invention for use in the repair or regeneration of a tissue selected from the group consisting of bone, soft tissue, cartilage, and brain or spinal cord tissue.

Another aspect of the present invention relates to the use of the engineered scaffold for bone repair or regeneration, such as the engineered scaffold for use in the repair and/or regeneration of brain or spinal cord tissue.

Yet another aspect of the present invention relates to the use of the engineered scaffold for cartilage tissue repair and/or regeneration.

Yet another aspect of the present invention relates to the tissue engineering of complete or parts of organs (e.g. liver, kidney, and lung), such as the engineered scaffold for use in the treatment of liver diseases, kidney diseases, lung diseases, bone diseases, cartilage diseases and/or other tissue diseases.

Another aspect of the present invention relates to the use of the engineered scaffolds for the cultivation of cells and/or bacteria.

Yet another aspect of the present invention relates to the use of the engineered scaffold for soft tissue repair and/or regeneration.

Still another aspect of the present invention relates to the use of the engineered scaffold for brain or spinal cord tissue repair and/or regeneration.

Another aspect of the present invention relates to the use of the engineered scaffold for the manufacture of a medicament, such as the engineered scaffold for use as a medicament.

Another aspect of the present invention related to a method of injecting the scaffold or subset of scaffolds of the present invention.

Yet another aspect of the present invention relates to the use of the engineered scaffold as a medicament.

Process for Preparing Scaffold

Throughout the recent years, an abundant number of processes have been suggested for the manufacturing of polymeric scaffolds, including solvent casting-particulate leaching, freeze-drying, rapid prototyping, electrospinning, and many more. The requirement for the process is that the produced scaffolds hold some general properties, such as biocompatibility, high porosity, biodegradability, and clinical handle ability.

Many scaffold fabrication techniques are not capable of precisely controlling pore size, and pore geometry, or the formation of channels within the scaffold. When applying for approval for clinical use, this may be critical, because a clear description of the product is required. However, in the present invention, high uniformity is present in the developed scaffolds, because the micro- and nano-structures of the freeze-dried polymer are highly reproducible and steerable, and because the plotted backbone determines the outer shape. The size and shape of the final scaffold is a matter of computer-aided design to print the wanted 3D template and adding the freeze-dried interior. The final scaffold can be understood as an open interconnected, hierarchically organised structure. At the micron to submicron length scale, the top/down manufacturing approach of rapid prototyping is used to make a structure that will constitute the frame into which the bottom/up processing approach of thermal induced phase separation form an open porous scaffold having a bimodal distribution of highly interconnected pores. In an embodiment, one set of pores 4a is above approximately 20 microns in size and the other set of pores 4b is below approximately 5 microns in size.

Thus, one aspect of the invention relates to a process for producing an injectable three-dimensional biocompatible scaffold 1 according to the invention, the process comprising:
a) providing a first biocompatible material 2,
   said first material is shaped as one or more grids forming an open network of first voids, said grid providing protective mechanical support for the second biocompatible material; wherein the grid has a diameter of less than 1.5 mm;
b) preferably, cooling the first biocompatible material to a temperature equal to or below 5° C.;
c) adding a solution comprising one or more biocompatible polymers and two or more solvents to a substantial part of the open network.
d) removing said solvents resulting in a second biocompatible material 3 within the open network,
   said second material filling being comprised in the first voids shaped by said first biocompatible material;
   said second biocompatible material comprising one or more biocompatible polymers;
   said second material being porous and the pores being interconnected;
   said second material having a plurality of open second voids 4a, 4b distributed therein, said open second voids being at least bimodal in size thereby providing voids which
      allows cells to, optionally infiltrate, grow and differentiate therein, and
      provide a stiffness of the second material different from the stiffness of the first biocompatible material.

In a preferred embodiment, the first biocompatible material is cooled to a temperature equal to or below 5° C. The present inventors have realized that by cooling the first biocompatible material during positioning of the second biocompatible material the degradation of the first biocompatible material (due to the solvent), is reduced to an acceptable level. Thus, the cooling steps protects the first biocompatible material from degradation, and thereby maintains the integrity of the first material and thus also the stiffness. In example 6, the stiffness of scaffolds has been determined with the different temperatures of the first material during step b).

Temperatures During Process for Preparing Scaffold.

As described above temperatures during the scaffold preparation process has great impact on the overall stiffness of small injectable scaffolds. Thus, in an embodiment, in step b) the first biocompatible material is cooled to a temperature below 0° C., such as below −10° C., such as below −20° C., such as below −40° C., such as below −80° C., such as below −120° C., or such as below −150° C. In another embodiment, in step b) the first biocompatible material is cooled to a temperature in the range 5 to −20° C., such as in the range −20 to −50° C., such as in the range −40 to −80° C., such as in the range −60 to −120° C., such as in the range −120 to −170° C., or such as in the range −150 to −210° C.

In yet another embodiment, in step b) the first biocompatible material is cooled to a temperature in the range 0° C. to −210° C., such as in the range 0 to −170° C., such as in the range 0 to −120° C. such as in the range −12 to −80° C., such as in the range 0 to −40° C., such as in the range 0 to −20° C., such as in the range 0 to −100° C., such as −20 to −100° C., such as −20 to −80° C., or such as in the range −80 to −210° C. In example 6, the temperature influence on the overall scaffold stiffness is described.

The temperature of the second material during the process may also influence both stiffness and pore sizes. Thus, in an embodiment, the solution added in step c) is added at a temperature above 5° C. such as above 12° C., such as in the range 5-25° C. or such as in the range 12-25° C. It is important that the temperature is not to low, since it influences on the phase separation of the solvents e.g. dioxane and water.

In another embodiment, the solution added in step c) is cooled during step c) to a first temperature matching the temperature of the first (cooled) material, thereby obtaining thermal equilibrium between the first material and the solution. In a further embodiment, the solution added in step c) is cooled during step c) to a first temperature in the range 5 to −20° C., such as in the range −20 to −50° C., such as in the range −40 to −80° C., such as in the range −60 to −120° C., such as in the range −120 to −170° C., or such as in the range −150 to −210° C.

In yet another embodiment, the solution is further cooled to a second temperature below the first temperature, such as to a temperature below −10° C., such as below −20° C., such as below −40° C., such as below −80° C., such as below −120° C., such as below −150° C. In a further embodiment, the temperature is kept at the first temperature range for a period of 1 to 30 minutes, such as 1 to 15 minutes, such as 1 to 10 minutes, before cooling to the second temperature.

Photocrosslinkable Polymers

In an embodiment of the invention, the process does not involve a photocrosslinkable polymer, such as

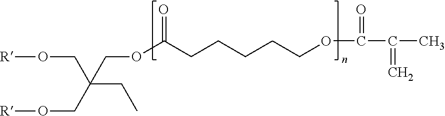

Photocrosslinkable polymers may be an attractive way to produce the first and/or the second biocompatible material according to the present invention. However, at the current stage the biocompatibility of such polymers are currently unknown. Since such photo-crosslinkable polymers may not be acceptable by e.g. the human body, it may therefore be desirable to avoid such polymers.

The grid may be produced by different means. Thus, in an embodiment the grid is produced by a process selected from the group consisting of solid freeform fabrication, rapid prototyping, salt leaching, fused deposition modelling and stereolithography. In one aspect of the present invention, the scaffold of the present invention may be replicated by two-photon polymerization, the principle disclosed in a paper by Ovsianikov et al. (Ultra-Low Shrinkage Hybrid Photosensitive Material for Two-Photon Polymerization Microfabrication.” ACS Nano 2 (11) (November 25): 2257-2262. 2008.

As previously mentioned the scaffold may be pre-seeded with cells. Thus, in an embodiment the process further comprises in vitro seeding the scaffold with cells.

The scaffold may also comprise one or more coating layers. Thus, in an embodiment the process further comprises:
a) coating the scaffold surface with one or more biocompatible polymers being negatively charged at physiological pH;
b) optionally, seeding the scaffold with cells;
c) optionally, coating the scaffold surface with one or more biocompatible polymers being positively charged at physiological pH.

In another embodiment, the process further comprises:
a) coating the scaffold surface with one or more biocompatible polymers being positively charged at physiological pH;
b) optionally, seeding the scaffold with cells;
c) optionally, coating the scaffold surface with one or more biocompatible polymers being negatively charged at physiological pH.

In a further embodiment, the scaffold surface is coated with a natural or synthetic coating material such as protein, peptides, nucleotides, and/or small interfering RNAs.

The mixture may comprise different solvents. In an embodiment, said mixture comprises three or more solvents. In another embodiment of the invention, the mixture comprises at least two solvents, such as within a range from about 3 to about 10 solvents, such as within a range from about 3 to about 5 solvents. Non-limiting examples of solvents are hexane, heptane, benzene, carbon tetrachloride, chloroform, acetic acid, ethyl acetate, THF, methylene chloride, acetone, ethanol, methanol, propanol, isopropanol, dioxane, acetonitrile, dimethylformamide, DMSO and water. In a specific embodiment, the mixture comprises dioxane and water.

The cavities in the scaffold may be produced in different ways. In an embodiment, the interconnected cavities are formed by thermal induced phase separation and/or lyophilisation of the solvents. In another embodiment, the interconnected cavities are formed by gas foaming. In a further embodiment the freezing points of the solvents are separated from each other by at least 5° C., such as within a range from about 5 to about 100 degree Celsius, such as within a range from about 6 to about 95 degree Celsius, such as within a range from about 8 to about 90 degree Celsius, such as within a range from about 10 to about 70 degree Celsius, preferably such as within a range from about 5 to about 100 degree Celsius.

In a preferred embodiment, the grid is produced by fused deposition modelling (FDM).

In another preferred embodiment the deposition of the first material is done at a temperature in the range 80-100° C., such as 90-100° C. or such as 90-95° C., preferably with the first material being PCL. These are process parameters employed in the example section.

In yet a preferred embodiment the temperature of the deposited material is lowered to below 40° C. between each deposition cycle, such as below 30° C., such as below 25° C., such as in the range 5-40° C., such as in the range 10-40° C., such as in the range 15-30° C., or such as in the range 15-25° C. As shown in the example section, this cooling process surprisingly improves the deposition process.

Similarly it has been observed that the deposition is improves when specific nozzles are employed. Thus, in an embodiment the diameter at the orifice of the nozzle of the depositing apparatus is in the range 0.1-0.2 mm. In yet an embodiment the area at the orifice of the nozzle of the depositing apparatus is in the range 0.00785-0.0314 mm².

Kit of Parts

The scaffold according to the present invention may be supplied as a kit. Thus, an aspect of the present invention relates to a kit comprising
a subset of scaffolds 1 according to the present invention; and
means for providing the scaffolds to a subject.

Such kit may make it easy for the user to insert the scaffold into a subject. In an embodiment, the means are a syringe having a needle diameter larger than the diameter of the scaffold. In yet an embodiment the means for providing the scaffolds to a subject is preloaded with the subset of scaffolds.

In Vitro Use

The scaffolds according to the present invention may find use ex vivo. Thus, an aspect of the present invention relates to the use of scaffolds 1 according to the present invention for in vitro seeding of cells. In yet an embodiment the invention relates to an engineered tissue made by contacting the biocompatible three-dimensional scaffold according to the present invention with cells in vivo or in vitro under conditions effective to allow interaction between the biocompatible three-dimensional scaffold and the cells. In certain embodiments, the cells are members selected from the group consisting of stem cells, progenitor cells, and/or differentiated cells, e.g. partially or fully differentiated cells. In another embodiment, the cells are members selected from the group consisting of regenerative cells. In certain embodiments, the cells are at least one of neural cells, epithelial cells, cardiac myocytes, pulmonary lung cells, keratinocytes, and endothelial cells. In certain embodiments, the cells are PC12 or neuronal-restricted precursor (NRP) cells and the engineered tissue is a neurone producing tissue.

Product Obtainable by Process

Another aspect relates to a scaffold according to the present invention obtainable/obtained by any of the above processes.

In sum, the present invention relates to a three-dimensional biocompatible scaffold capable of supporting cell activities, such as growth and differentiation, the scaffold comprising a first biocompatible material and a second biocompatible material, said second material filling a substantial part of the first voids shaped by said first biocompatible material, wherein the scaffold has a diameter of less than 1500 μm.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

### Manufacturing of Injectable Scaffolds

In examples 1-3 Fused Deposition Modelling (FDM) has been used for the production of Hierarchical Scaffolds as Injectables (HSI).

The FDM machine used to perform the initial tests was of the brand SysEng Bioplotter. This machine deposits material by extruding the material from the nozzle. The material is heated to above its glass transaction temperature, after which it is deposited.

For the FDM procedure PCL has been tested but other materials may be used.

FIG. 1 shows a schematic overview of an injectable scaffold 1 according to one embodiment of the invention. It is to be understood that the first material 2 forms the overall structure and protects the scaffold against external forces. The second material 3 provides a stiffness, which allows cells to differentiate into the desired cell type. The larger pores 4a in the second material provide space for living cells, and the smaller pores 4b provides space for diffusion of nutrients and the like. Thus, in FIG. 1 the white space inside the scaffold (but outside the circles 4a, 4b) illustrates the second material 3. The thinner black lines inside the scaffold simply illustrates that the second material is formed by polymers. The dotted outer line indicates that the scaffold is permeable, porous and diffusible by nutrients.

### Example 2

### Optimizing FDM Procedure—Temperature

The viscosity of the polymeric material can be controlled by controlling the nozzle temperature. The position of the nozzle is controlled by using CNC.

By building topics by conventional settings on FDM machine (nozzle temperature=106° C.), it has not been possible to manufacture a geometry (first biocompatible material according to the invention) of the desired size (data not shown).

To see whether change in temperature could improve the manufactured structures a lower die temperature of between 90-95° C. were tested. However, when lowering the temperature the viscosity increases. The increased viscosity provides the following challenge. 1) The delay of the material exiting the extruder, caused by a higher viscosity, resulted in damaged structures (data not shown). To overcome the viscosity problem a start signal was provided to the machine before the nozzle is at the desired geometry position. In addition, a stop signal was provided before the machine had finished the geometry. In this way, it has been possible to build the desired geometry (first biocompatible material of the invention). In addition to this, it has been found to be necessary for each layer to cool completely at room temperature before the next layer is deposited.

### Example 3

### Optimizing FDM Procedure—Nozzle Size (Area/Orifice of the Nozzle)

Different nozzle sizes have been tested for the process according to the invention. By using a nozzle with a diameter of 0.1 mm to 0.2 mm (diameter in the range 0.00785-0.0314 mm²). A higher geometric accuracy can be achieved compared with this range compared to nozzles with larger holes. Deposition of material by lower nozzle diameter is made difficult by the shear stresses which arise at the interface between the material and the die (data not shown). For this reason nozzle temperatures are often used which are higher than the glass transition temperature of the material.

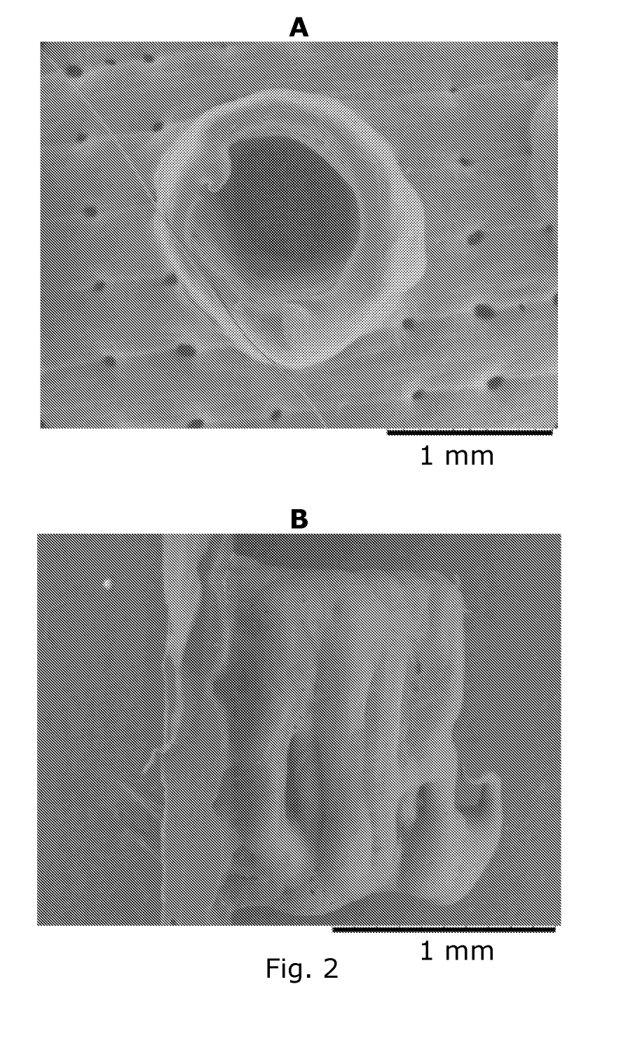

Pictures of FDM produced geometries are shown in FIG. 2.

### Example 4

### Thermal Induced Phase Separation (TIPS)—Nano Foam Generation

In the present example the solution is composed of 1,4-Dioxane, milliQ water and polycaprolactone (PCL), commercial grade 6405th

The ratio between the ingredients is crucial for the properties and topology of the TIPS material. There is in principle three parameters to adjust when generating TIPS material.
a. The molecular weight of PCL.
b. The amount of PCL to water and dioxane
c. The temperature gradient employed during cooling.

To produce the optimal inner material of the scaffold these three parameters most be mutually adjusted. By controlling the cooling rate, the phase separation of dioxane and water is also controlled. In this way, the pore sized in the TIPS material may be varied (second biocompatible material of the invention).

The melting point of water is at 0° C. After the dissolution passes the 0 point, the water will precipitate crystals. Water and PCL are not present inside the dioxane, but are diffused into the interface. Here the water will precipitate crystals that take up space. Thus, rapid cooling gives more and smaller crystals. By adding more water to the solution, it is possible to control the number and size of small porosities.

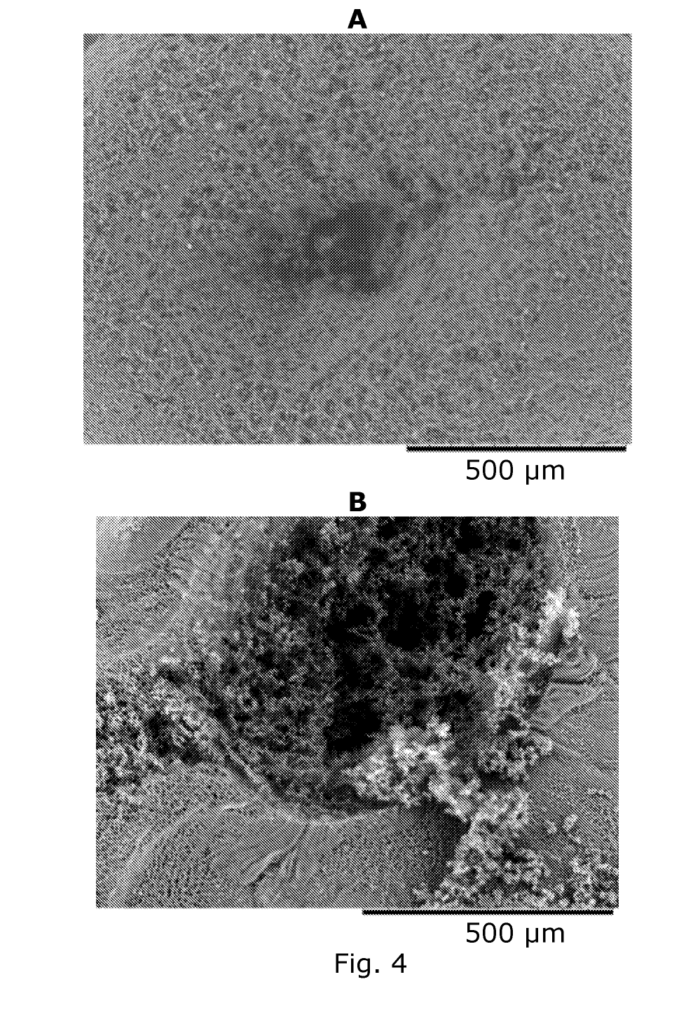

The cooling process may be performed in a dedicated cooling chamber, e.g. containing liquid nitrogen, to control the temperature gradient. FIG. 3A shows a diagram with different temperature cooling profiles. FIG. 3B shows the stiffness in kPa for the second biomaterial 3 as a function of solvent composition FIG. 4 shows two different scaffolds produced by two different cooling gradients.

After the TIPS solution is cooled, the subject is transferred to a freeze dryer. Topics may be dried at −40° C. for 24 hours.

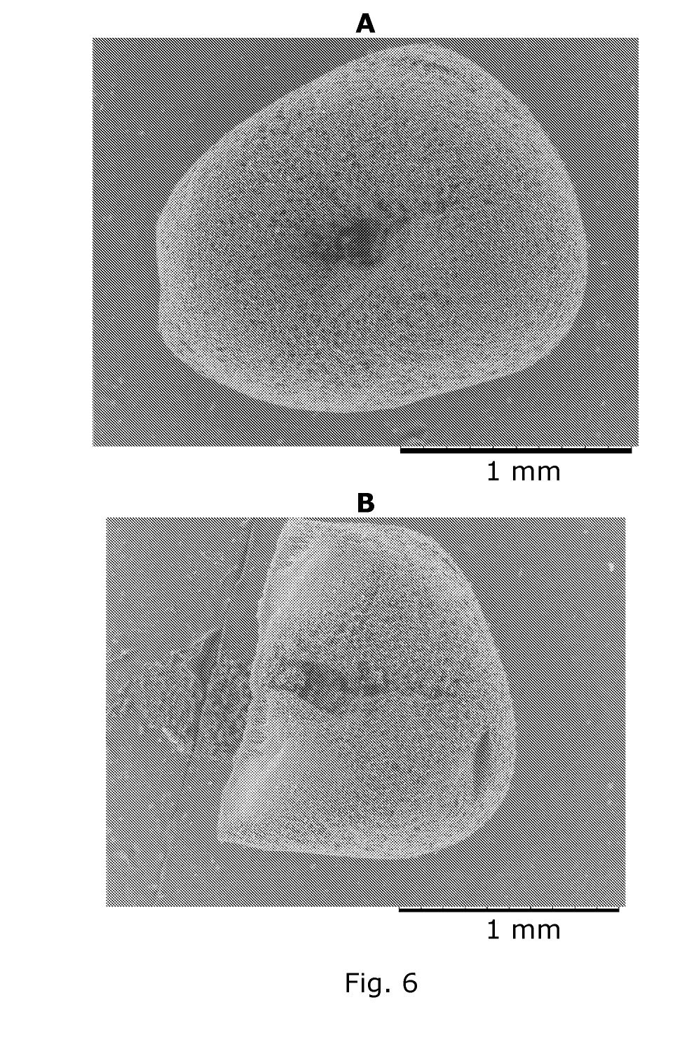

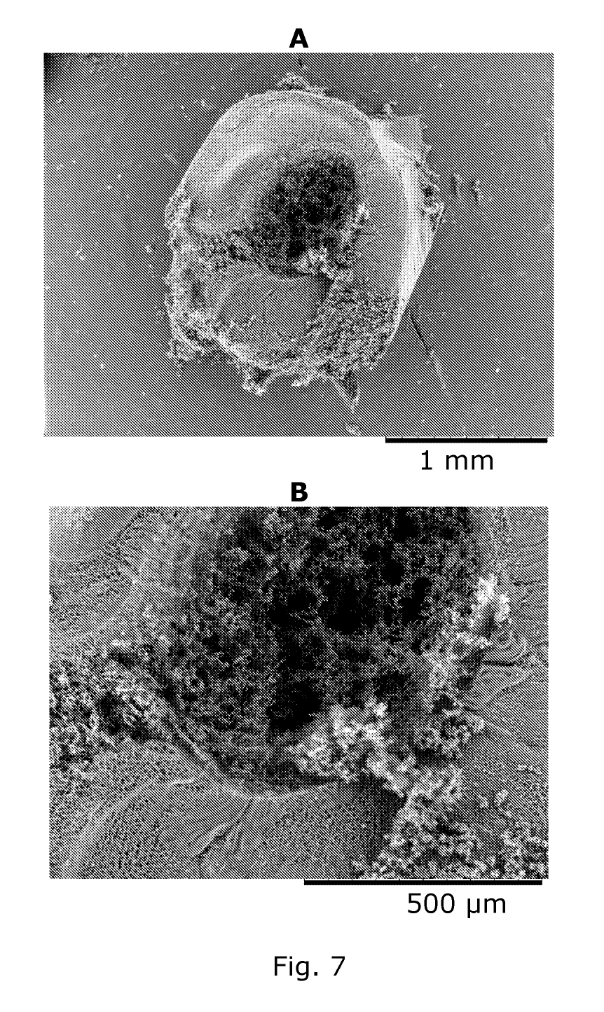

The dried matter is transferred to a petri dish. The FDM produced geometry is now fully embedded in the dried TIPS foam. In order to separate the item (FDM with TIPS incorporated) from the excess TIPS material the Petri dish is mounted on a cyclone stirrer at 800 rpm for approx. 10 min. After this treatment, the product is separated from the excess material. FIGS. 5-7 shows examples of the finished product (HSI) (three-dimensional biocompatible scaffold of the invention).

Conclusion

The method of production, results in an injectable scaffold given by the combination of FDM and TIPS. The FDM-produced component of the product is decisive for the overall exterior geometry of the product.

The TIPS component is decisive for the inner (core) of the product.

It is in earlier work demonstrated that topology and mechanical properties of TIPS component can be varied and controlled within narrower frames. The present invention has refined the process allowing for the production of hierarchical structures with a physical size permitting the structures to be injected, e.g. with an injection needle.

### Example 5

An embodiment of the process for producing scaffolds according to the present invention.

A cooled micro-mould was designed to contain particles of the first material. The mould and its content is cooled to −20° C. and thermal equilibrium is obtained. A solution of the second material is prepared and maintained at 20° C. The solution at 20° C. is injected into the mould. The combined structures and liquids is immediately cooled to −20° C. and thermal equilibrium is obtained at −20° C. After one minute, the temperature of the assembly is further lowered to −60° C.

### Example 6

Comparison of stiffness of injectable scaffolds depending on temperature of the first material during production.

Scaffolds were produced according to process described in example 5, with the difference that the temperature of the first material was varied. The temperature of the first material was adjusted as shown in the table below.

Results

| Temperature of first material | |
|---|---|
| during production | Compression stiffness of scaffold |
| 20° C. | − |
| 0° C. | + |
| −20° C. | ++ |
| −50° C. | +++ |
| −120° C. | ++++ |

### CONCLUSION

As shown in the above table the temperature of the first material during production has a great influence of the overall strength of the first material according to the invention.

As previously mentioned, it is believed that the solvent comprising the second material is less aggressive on the first material when said first material has been cooled. This is also important knowledge when the strength of the first material is adapted to match the stiffness of the tissue in which the specific scaffold is to be injected in.

1. A three-dimensional biocompatible scaffold comprising:
a first biocompatible material and a second biocompatible material,
said first biocompatible material being shaped as one or more grids forming an open network of first voids, said one or more grids being configured to provide a protective mechanical support for said second biocompatible material;
said second biocompatible material being in the first voids shaped by said first biocompatible material;
said second biocompatible material comprising one or more biocompatible polymers;
said second biocompatible material being porous and having pores that are interconnected; and
said second biocompatible material having a plurality of open second voids distributed therein, said open second voids being at least bimodal in size distribution, thereby providing voids which:
allow cells to, optionally infiltrate, grow and differentiate therein, and
provide a stiffness to the second biocompatible material different from the stiffness of the first biocompatible material;
wherein the three-dimensional biocompatible scaffold has a diameter of less than 1500 μm.

2-42. (canceled)

43. The three-dimensional biocompatible scaffold according to claim 1, wherein the first biocompatible material has a value of compression stiffness comparable to the value of compression stiffness of the surrounding tissue after insertion in a body.

44. The three-dimensional biocompatible scaffold according to claim 1, wherein the first biocompatible material has a compression stiffness configured to withstand compressive forces from surrounding targeted tissue after insertion in a body.

45. The three-dimensional biocompatible scaffold according to claim 1, wherein the first biocompatible material has a compression stiffness in the range of 0.3-100 kPa and the second biocompatible material has a compression stiffness in the range of 0.3-100 kPa.

46. The three-dimensional biocompatible scaffold according to claim 1, wherein the first biocompatible material of the three-dimensional biocompatible scaffold has a compression stiffness in the range of 60 kPa-2 MPa and the second biocompatible material has a compression stiffness in the range of 0.3-100 kPa.

47. The three-dimensional biocompatible scaffold according to claim 1, wherein the first biocompatible material of the three-dimensional biocompatible scaffold has a compression stiffness in the range of 2-1000 MPa and the second biocompatible material has a compression stiffness in the range of 0.3-100 kPa.

48. The three-dimensional biocompatible scaffold according to claim 1, wherein the first biocompatible material of the three-dimensional biocompatible scaffold has a compression stiffness in the range of 1-25 GPa and the second biocompatible material has a compression stiffness in the range of 0.3-100 kPa.

49. The three-dimensional biocompatible scaffold according to claim 1, wherein the three-dimensional biocompatible scaffold is biodegradable.

50. The three-dimensional biocompatible scaffold according to claim 1, wherein the three-dimensional biocompatible scaffold further comprises living cells.

51. The three-dimensional biocompatible scaffold according to claim 1, wherein the average size of larger pores of said open second voids that are bimodal in size distribution are in the range 75 to 800 μm and average size of smaller pores of said open second voids that are bimodal in size distribution are in the range 0.01-10 μm.

52. The three-dimensional biocompatible scaffold according to claim 1, wherein the first biocompatible material comprises a material selected from the group consisting of PCL, PET, PC, PEEK, PP, PE, and derivatives thereof.

53. The three-dimensional biocompatible scaffold according to claim 1, wherein the second biocompatible material comprises a material selected from the group consisting of Polyethyleglycol (PEG), Polylacticglycolacid (PLGA), Poly Lactic Acid (PLA), Poly Lactic Lactic Acid (PLLA), Polycaprolactone (PCL), Polyethyleterapthalate (PET), Polycarbonate (PC), Polyetheretherketone (PEEK), Polypropylene (PP), Polyethylene (PE), and derivatives thereof.

54. The three-dimensional biocompatible scaffold according to claim 1, wherein the first and the second biocompatible material comprise PCL or the first and the second biocompatible material consist of PCL.

55. A process for producing an injectable three-dimensional biocompatible scaffold according to claim 1, the process comprising:
a) providing a first biocompatible material,
said first biocompatible material being shaped as one or more grids forming an open network of first voids, said one or more grids providing protective mechanical support for a second biocompatible material; wherein said one or more grids has a diameter of less than 1500 μm;
b) cooling the first biocompatible material to a temperature equal to or below 5° C.;
c) adding a solution comprising one or more biocompatible polymers and two or more solvents to a substantial part of the cooled open network;
d) removing said solvents thereby generating a second biocompatible material within the open network,
said second biocompatible material being in the first voids shaped by said first biocompatible material;
said second biocompatible material comprising one or more biocompatible polymers;
said second biocompatible material being porous and having pores that are interconnected; and
said second biocompatible material having a plurality of open second voids distributed therein, said open second voids being at least bimodal in size thereby providing voids which:
allow cells to, optionally infiltrate, grow and differentiate therein, and
provide a stiffness to the second biocompatible material different from the stiffness of the first biocompatible material.

56. The process according to claim 55, wherein the one or more grids is produced by a process selected from the group consisting of solid freeform fabrication, rapid prototyping, fused deposition modelling (FDM), and stereolithography.

57. The process according to claim 55, wherein the deposition of the first material is done at a temperature in the range 80-100° C.

58. The process according to claim 55, wherein in step b) the first biocompatible material is cooled to a temperature below 0° C.

59. A kit comprising
a subset of scaffolds according to claim 1; and
a delivery system.

60. The kit according to claim 59, wherein said delivery system is one or more syringes having a needle diameter larger than the diameter of the scaffold.
